(19) United States
(12) Patent Application Publication
(10) Pub. No.: US 2024/0327513 A1
(43) Pub. Date: Oct. 3, 2024

(54) BINDING-TRIGGERED REGULATION OF PROTEIN DEGRADATION

(71) Applicant: The Regents of the University of California, Oakland, CA (US)

(72) Inventors: Andrew H. NG, San Francisco, CA (US); Matthew KIM, San Francisco, CA (US); Wendell A. LIM, San Francisco, CA (US); Hana EL-SAMAD, San Francisco, CA (US)

(21) Appl. No.: 18/278,379

(22) PCT Filed: Mar. 14, 2022
(86) PCT No.: PCT/US2022/020214

§ 371 Date: Aug. 22, 2023

Related U.S. Application Data
(60) Provisional application No. 63/161,312, filed on Mar. 15, 2021.

(51) Int. Cl. C07K 16/28; C07K 14/395; C12N 5/0783; C12N 15/86

(52) U.S. Cl. CPC C07K 16/2809; C07K 14/395; C12N 5/0636; C12N 15/86; C07K 2319/03; C07K 2319/30; C07K 2319/74; C12N 2740/15043

(57) ABSTRACT

Provided herein is cleavable fusion protein comprising: an extracellular binding domain comprising a first protein binding domain that specifically binds to a cell surface protein, a transmembrane domain, one or more force-dependent cleavage sites and an intracellular domain comprising: i. a second protein binding domain that specifically binds to a target protein; and ii. a degradation domain. When the fusion protein is expressed in a mammalian cell, binding of the first binding domain to the cell surface protein induces proteolytic cleavage of the one or more force-dependent cleavage site to release the intracellular domain. Protein circuits, cells and methods that make use of the fusion protein are also provided.

### CROSS-REFERENCING

This application claims the benefit of U.S. provisional application Ser. No. 63/161,312, filed on Mar. 15, 2021, which application is incorporated by reference herein.

### GOVERNMENT RIGHTS

This invention was made with government support under grant no. HR0011-16-2-0045 awarded by Defense Advanced Research Projects Agency. The government has certain rights in the invention.

### BACKGROUND

Regulating the activity of specific proteins inside a cell using an external stimulus is a central challenge to cell engineering. Existing methods largely focus on regulating gene expression via small molecules. However, small molecule approaches are only really limited to certain target proteins. In addition, the small molecule used may be toxic to the body, limiting their use in therapeutic applications. In many cell engineering applications such as CAR T cell therapy and stem cell therapies, it would be advantageous to genetically encode a way for a target protein to be regulated by an environmental signal, without a small molecule input. This disclosure addresses this need and others.

### SUMMARY

This disclosure provides a way to activate or deactivate protein degradation in a cell in response to an antigen binding event on the outside of the cell. The method makes use of a fusion protein that contains an extracellular binding domain (such as a scFv) linked to an intracellular degradation domain and another protein binding domain, separated by a transmembrane domain and one or more force-dependent cleavage sites that are cleaved when the extracellular binding domain binds to its target on another cell. The combined protein degradation domain and protein binding domain on the intracellular side of the fusion may be referred to as a “synthetic targeter of ubiquitination and degradation”, or “STUD” for short in this disclosure. A signal sequence fused to the extracellular domain traffics and localizes the STUD to the plasma membrane, simultaneously targeting a target protein that is at the plasma membrane for degradation and preventing degradation of a cytosolic or nuclear target protein. Binding of the extracellular binding domain to a target antigen displayed on an opposing cell or any surface, triggers proteolytic cleavage of force-dependent cleavage sites, releasing the STUD from the plasma membrane. This simultaneously deactivates targeted degradation of a membrane protein target and enables degradation of a cytosolic or nuclear target protein.

Specifically, if the STUD targets degradation of a protein that is localized to the plasma membrane, then binding of the extracellular binding domain to a target antigen displayed on an opposing cell decreases degradation of the protein. In contrast, if the STUD targets degradation of a cytoplasmic or nuclear protein, then binding of the extracellular binding domain to a target antigen displayed on an opposing cell increases degradation of the cytoplasmic or nuclear protein because the STUD has been released from the plasma membrane and can diffuse to those proteins. Depending on the targeting domain of the fusion protein, the fusion protein can be used to degrade transmembrane receptors (e.g., chimeric antigen receptors) or intracellular targets (e.g., kinases/phosphatases and/or synthetic/endogenous transcription factors) in an antigen-dependent manner. As will be described in greater detail below, this fusion protein enables “AND” and “OR” logic-gated T cell activation by stabilizing a CAR in the presence of a target antigen. In this example, the extracellular binding domain of the fusion protein may be a first cancer antigen and the intracellular binding domain may target a CAR for degradation, where the CAR binds to a second cancer antigen. In this example, if both the fusion protein and the CAR of the same cell bind to antigens on one or more other cells, then the STUD should be released from the plasma membrane and the CAR will be degraded at a reduced rate. This circuit reduces or prevents off-tumor toxicity of cell therapies. Likewise, the system can be used to manipulate transcription factors that are involved in cell differentiation, for example.

In more detail, the fusion protein provides a way to degrade proteins in an antigen dependent manner. In one example, the protein can be used to regulate degradation of cytosolic or nuclear protein targets. In this example, while the intracellular STUD is held at the membrane, it is unable to interact with proteins in the cytosol or nucleus. Once the intracellular STUD is released from the membrane after antigen engagement and subsequent proteolytic cleavage of the cleavage sites, the STUD can then bind to and degrade cytosolic or nuclear proteins. These proteins could include important endogenous signaling molecules such as transcription factors, phosphatases or kinases. For example, bZIP transcription factors (involved in cell fate decision making, and importantly implicated in T cell exhaustion) and Ras-Raf-Mek MAPK signaling proteins could be targeted.

In another example, the fusion protein can be used to regulate the degradation of a synthetic membrane protein such as CAR or SynNotch receptor or an endogenous membrane receptor such as PD-1 or a TCR based on the binding domain of the STUD. In the absence of the target antigen in the other cell, the fusion protein should target the target membrane protein for degradation. Once the fusion protein binds its cognate antigen on another cell, the STUD will be released from the membrane and will no longer be able to degrade its target membrane protein. This embodiment enables combinatorial antigen recognition, where the targeted receptor will be conditionally stabilized only in the presence of the cell surface antigen recognized by the fusion protein.

In some embodiments, the cleavable fusion protein may contain: (a) an extracellular binding domain comprising a first protein binding domain, wherein the first protein binding domain specifically binds to a cell surface protein; (b) a force sensing region; (c) a transmembrane domain; (d) one or more force-dependent cleavage sites that are cleaved when the force sensing region is activated; and (e) an intracellular domain comprising: i. a second protein binding domain, wherein the second protein binding domain specifically binds to a target protein; and ii. a degradation domain, wherein the degradation domain is a degron or E3 ligase-recruiting domain, and when the fusion protein is expressed in a mammalian cell, binding of the first binding domain to the cell surface protein induces proteolytic cleavage of the one or more force-dependent cleavage sites to release the intracellular domain.

Cells and protein circuits containing the fusion protein and the protein targeted by the STUD are also provided. In these embodiments, the cell or protein circuit may comprise a fusion protein as described above and the target protein, where binding of the extracellular domain of the fusion protein to a cell surface protein on another cell releases the intracellular domain and alters degradation of the target protein. Degradation of the target protein may be increased or decreased, depending on whether the target protein is localized to the plasma membrane or is not localized to the plasma membrane.

A method for modulating degradation of a target protein is also provided. In some embodiments this method may comprise: introducing a first cell to a second cell, wherein: (a) the first cell comprises the fusion protein of claim 1-6 and the target protein; and (b) the second cell comprises the cell surface protein on its surface. In this method, binding of the first cell to the second cell via the fusion protein releases the intracellular domain, thereby altering degradation of the target protein. Again, degradation of the target protein may be increased or decreased, depending on whether the target protein is localized to the plasma membrane or is not localized to the plasma membrane.

These and other advantages may become apparent in view of the following discussion.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates an example of the present fusion protein.

FIGS. 2A-2C schematically illustrates the principle upon which some embodiments of the present method work. FIG. 2A is an illustration showing that how the present fusion protein cleaves a target protein in a binding-dependent manner. FIG. 2B is an illustration how the present fusion protein can be used to deactivate or activate transcription. FIG. 2C illustrates how the fusion protein can be used as part of an AND gate to effect killing of a cell only if the cell expresses two particular cell surface antigens, one recognized by the fusion protein the other recognized by a chimeric immunoreceptor (e.g., a chimeric antigen receptor).

FIG. 3: Lysine to arginine substitution significantly improves STUD activity. Either a GFP nanobody (vhhGFP4) or SynZIP (SZ18) were used to target a GFP (or in the case of the SZ18 STUD, GFP-SZ17. SZ17 and SZ18 form a cognate pair). GFP % Degradation was measured compared to GFP fluorescence in the absence of the STUD.

FIG. 4: MG132 proteasome inhibitor confirms that the effect of STUD is mediated by the proteasome. Primary human CD4+ T cells expressing different variants of the GFP nanobody STUD were treated with 5 uM MG132 and fluorescence was measured at 1 and 3 hours post induction. The mutant nanobody was the only experimental group that exhibited an increase in fluorescence over time, suggesting the effect of the STUD is mediated by protein degradation through the proteasome.

FIG. 5: Optimizing STUD activity via linker modification in Jurkat cells. A variety of flexible (GS) and rigid linkers were tested between the SynZIP targeting domain and degron on the STUD. Flexible linkers generally outperformed rigid linkers, and in particular the 5xGS linker produced the greatest degradation.

FIG. 6: Design of a circuit to test STUD induced degradation of a synthetic transcription factor. VPR-NS3-ZF3 drives activation of the pZF3(8x)_ybTATA promoter in response to induction with the small molecule Grazoprevir (GRZ). Three different circuit configurations were explored. This circuit provides feedback, where STUD is driven off the pZF3 promoter, GFP alone, where no STUD is expressed, and constitutively expressed STUD, where the STUD is expressed off the pPGK promoter

FIG. 7: ZF3 circuit dose responses demonstrate the functionality of the soluble STUD to degrade a transcription factor. The circuits shown in FIG. 3 were transduced into Jurkat cells and induced with a range of GRZ concentrations to activate the TF. GFP fluorescence was measured 72 hours later.

FIG. 8: Testing the ability of soluble STUDs to target a CAR-SZ17 fusion for degradation in Jurkat cells. Four different linker lengths between the SynZIP18 on the STUD and degron were tested. A control where the degron was directly fused to the CAR generated the most degradation. These results highlight the ability of our specific degradation pathway to control CAR expression in T cells.

FIG. 9: Design of membrane targeting STUD. DAP10 extracellular domain (ECD) contains a signal sequence that traffics the protein in the membrane. The CD8 transmembrane domain (TMD) embeds into the membrane and is linked to the soluble STUD via a linker.

FIG. 10: Degradation of CAR in primary human CD4+ T cells. Rigid15 linker between CD8 TMD and soluble STUD mediated the greatest amount of CAR degradation as measured by staining for the myc-tag present on the extracellular domain of the CAR and flow cytometry.

FIG. 11: Degradation of SynNotch in primary human CD4+ T cells. Rigid15 linker between CD8 TMD and soluble STUD mediated the greatest amount of SynNotch degradation as measured by staining for the myc-tag present on the SynNotch and flow cytometry.

FIGS. 12A and 12B. Design of synthetic SynNotch receptor allows for antigen triggered degradation of cytosolic proteins. (12A) Schematic illustration of a NotchSTUD synthetic receptor that couples antigen binding to target degradation. (12B) Data showing that construct works as predicted.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

The term “binding” refers to a direct association between two molecules, due to, for example, covalent, electrostatic, hydrophobic, and ionic and/or hydrogen-bond interactions, including interactions such as salt bridges and water bridges. In some cases, the first member of a specific binding pair present in the extracellular domain of a chimeric Notch receptor polypeptide of the present disclosure binds specifically to a second member of the specific binding pair.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014)65:333-47; Sadelain et al. Cancer Discov (2013) 3(4):388-98; Cartellieri et al., J Biomed Biotechnol (2010) 956304; the disclosures of which are incorporated herein by reference in their entirety.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

The term “marker” refers to a protein that is on the surface of another cell. A marker may be a cell surface receptor, an epitope in a cell surface protein or a cell-surface ligand.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As noted above, this disclosure provides a cleavable fusion protein comprising: an extracellular binding domain comprising a first protein binding domain that specifically binds to a cell surface protein, a transmembrane domain, one or more force-dependent cleavage sites and an intracellular domain comprising: i. a second protein binding domain that specifically binds to a target protein; and ii. a degradation domain. The force-dependent cleavage sites are between the extracellular binding domain and the intracellular domain. Binding of the first binding domain to the cell surface protein induces proteolytic cleavage of the one or more force-dependent cleavage sites to release the intracellular domain. Protein circuits, cells and methods that make use of the fusion protein are also provided.

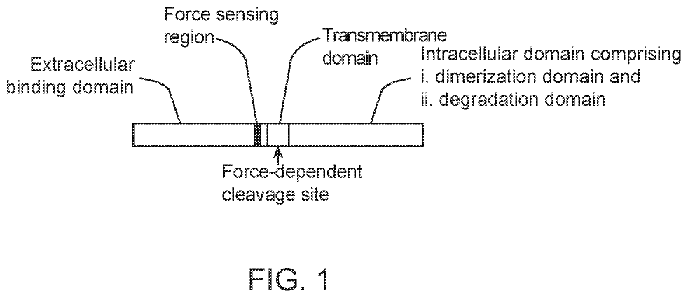

An exemplary fusion protein is schematically illustrated in FIG. 1. As shown, in some embodiments, the cleavable fusion protein may comprise: (a) an extracellular binding domain comprising a first protein binding domain, wherein the first protein binding domain specifically binds to a cell surface protein; (b) a force sensing region; (c) a transmembrane domain; (d) one or more force-dependent cleavage sites that are cleaved when the force sensing region is activated; and (e) an intracellular domain comprising: i. a second protein binding domain, wherein the second protein binding domain specifically binds to a target protein; and ii. a degradation domain, wherein the degradation domain is a degron or E3 ligase-recruiting domain. When the fusion protein is expressed in a mammalian cell, binding of the first binding domain to the cell surface protein induces proteolytic cleavage of the one or more force-dependent cleavage sites to release the intracellular domain.

In any embodiment, the fusion protein may optionally contain a linker any of the component parts, e.g., the second protein binding domain and the degradation domain. In a cell, binding of the fusion protein a target protein on another cell (via the first protein binding domain) releases the intracellular domain. The intracellular domain binds to a target protein via the second protein binding domain and induces degradation of the target protein.

Degradation may be ubiquitination-mediated or not ubiquitination-mediated, depending on which degradation domain is used. The various components parts of the fusion protein are described below.

### First Binding Domains

The extracellular domain of the fusion protein comprises a first protein binding domain that specifically binds to a cell surface protein. In some embodiments, the first protein binding domain could be a scFv, a nanobody (i.e., a single chain antibody derived from a camel or shark antibody variable domain), or a ligand for a cell-surface receptor, although other types of binding domains can be used in certain circumstances. For example, a cAb VHH (camelid antibody variable domain), IgNAR VH (shark antibody variable domain) and/or sdAb VH (single domain antibody variable domain) and “camelized” antibody variable domains (including humanized versions of the same) could be employed. First protein binding domains include, for example, antibody binding domains that bind to cell surface antigens, ligands that bind to cell surface receptors and receptors that bind to ligands. Other types of binding domains could be used in certain cases.

As will be apparent from the description that follows below, the cell surface marker to which the first protein binding domain binds may vary depending and how the fusion protein is going to be used.

### Transmembrane Domains

The fusion protein has a transmembrane domain. Suitable transmembrane domains include those of CD8, CD4, CD3 zeta, CD28, CD134, CD7, although there are thousands of others that one could use. As would be apparent, the nucleic acid encoding such a fusion protein may additionally comprise a signal peptide. In some embodiments, a transmembrane segment can comprise an amino acid sequence having at least 75%, at least 80%, at least 85%, at least 90%, at least 95%, at least 98%, at least 99%, or 100%, amino acid sequence identity to the following amino acid sequence: HLMYVAAAAFVLLFFVGCGVLLS (SEQ ID NO:1); and can have a length of 21, 22, 23, 24, or 25 amino acids.

### Linkers

In some embodiments, the fusion protein may further comprise a linker, between any two component parts of the fusion protein. A peptide linker can vary in length from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

Suitable linkers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary linkers include glycine polymers (G)n, glycine-serine polymers, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine and is much less restricted than residues with longer side chains (see Scheraga, Rev. Computational Chem. 11173-142 (1992)).

### Second Protein Binding Domains

The second protein binding domain of the fusion protein (i.e., the intracellular protein binding domain) may be any natural or engineered (non-natural) domain that specifically binds to a target protein.

In some embodiments, the second protein binding domain may be derived from an antibody. In these embodiments, the second protein binding domain may be a scFvs, nanobodies or intrabody, or the like. In other embodiments, the second protein binding domain is not an antibody-based protein binding domain. In these embodiments, the second protein binding domain may be a helix-turn-helix-based designed heterodimers (DHDs) described in Chen et al (Nature. 2019 565: 106-111), the heterospecific synthetic coiled-coil synthetic leucine zippers (synZIPs) described in, e.g., Thompson et al. (ACS Synth. Biol. 2012 1: 118-29), Reinke et al. (JACS 2010 132:6025-31) and (Cho et al Cell 2018 173: 1426-1438), miniproteins (Nature 2017 550: 74-79), intrabodies (Chen et al Human Gene Therapy 1994 5: 595-601). DARPins, an SH2 domain, an SH3 domain, or a PDZ domain could also be used in some cases, among many others. For example, if synZIPs are used, one dimerization domain may be BZip (RR) and the other one may be AZip (EE). SYNZIP 1 to SYNZIP 48, and BATF, FOS, ATF4, ATF3, BACH1, JUND, NFE2L3, and HEPTAD may be used in some cases. In some embodiments, a second protein binding domain could be designed using the Rosetta program described in Kuhlman et al (J. Biol. Chem. 2019 294, 19436-19443 and Leman et al 9 Nat. Methods 2020 17: 665-680). A “designed heterodimer”, i.e. a helix-loop-helix bundles described in Chen et al (Nature 2019 565: 106-111) and US20210355175A1 could also be used.

In some embodiments, the second protein binding domain may bind to its target orthogonally, meaning that it does not detectably bind to other proteins in the cell.

The proteins to which the second protein binding domains bind, i.e., the target proteins, are described in greater detail below.

### Degrons

Degrons are relatively short (typically under 100 amino acids) sequences that, when they are present in a protein, target that protein for degradation. Degrons include ubiquitin-dependent degrons and ubiquitin-independent degrons. Examples of degrons include ubiquitin (which is approximately 76 amino acids in length), PEST sequences (which are approximately 10 to 60 amino acids in length and are rich in P (proline), E (glutamate), S (serine), and T (threonine)), N-degrons (which are short N-terminal sequences), C degrons (which are short C-terminal sequences), unstructured initiation sites and short sequences rich in acceptor lysines. Degrons are diverse in sequence and have been extensively reviewed (see, e.g., Varshavsky, Proc. Natl. Acad. Sci. 2019 116: 358-366; Varshavsky, Protein Sci. 2011 20: 1298-1345; Natsume et al., Annu Rev. Genet 2017 51: 83-102; Rechsteiner et al., Trends Biochem Sci. 1996 21: 267-271; Herbst et al., Oncogene 2004 23: 3863-3871; Prakash, Nat. Struct. Mol. Biol. 2004 11: 830-837; Guharoy et al., Nat. Commun. 2016 7: 10239 and Chassin et al. Nature Comm. 2019 10).

The “Bonger”-type degron (which is composed of the sequence RRRG, where the G is the C terminus or one, amino acid away from the C terminus) may be used in any fusion protein, although there are many alternatives that could be used instead.

Examples of C-degrons suitable for use in a fusion protein are listed below (see Koren et al, Cell 2018 173: 1622-1635):

| | | | SEQ ID |
|---|---|---|---|
| Name | Sequence | Motif | NO: |
| fRA68_EMID1 | RGKRGGHATNYRIVAPRSRDERG* | RG* | 2 |
| fRA69_CHGA | ESLSAIEAELEKVAHQLQALRRG* | RG* | 3 |
| FRA70_MAGEA3 | KISGGPHISYPPLHEWVLREGEE* | EE* | 4 |
| fRA71_MAGEA3EEtoAA | KISGGPHISYPP LHEWVLREGAA* | EE* to | 5 |
| | | Ax/A* | |
| FRA72_PIK3C2B | LRELDLAQEKTGWFALGSRSHGTL* | RxxGxx* | 6 |
| FRA73_PXN | LRELDLAQEKTGWFALGSRHCGRT* | RxxGxx* | 7 |
| fRA74_Peptide35 | YKKAGSGIPLRMNSLFRKRNKGK* | RxxGxx* | 8 |
| fRA75_CDK5R1 | VFSDLKNESGQEDKKRLLLGLDR* | R* motif, | 9 |
| ERA76_CDK5R1trunc | VFSDLKNESGQEDKKRLLLGLD* | R | 10 |
| | | truncated, | |
| fRA77_SIL1 | DGEDEGYFQELLGSVNSLLKELR* | R* | 11 |
| fRA78_SIL1trunc | DGEDEGYFQELLGSVNSLLKEL* | R | 12 |
| | | truncated, | |
| fRA79_N-Myc | LEKEKLQARQQQLLKKIEHARTC* | Rxx* | 13 |
| FRA80_N-Myctrunc | LEKEKLQARQQQLLKKIEHA* | Rxx* to | 14 |
| | | A* | |
| fRA81_MSRB2 | GPGPNGQRFCINSAALKFKPRKH* | Rxx* | 15 |
| fRA82_OR4C13 | LRNAQMKNAIRKLCSRKAISSVK* | Vx* motif | 16 |
| ERA83_OR4C13Dmut | LRNAQMKNAIRKLCSRKAISSDK* | Vx to Dx | 17 |
| fRA84_SREBF2 | RRSCNDCQQMIVKLGGGTAIAAS* | Ax* | 18 |
| fRA85_SREBF2-Dmut | RRSCNDCQQMIVKLGGGTAIADS* | Ax | 19 |
| fRA86_CPS1 | QKSRKVDSKSLFHYRQYSAGKAA* | AA* | 20 |
| FRA87_CPS1DDmut | QKSRKVDSKSLFHYRQYSAGKDD* | AA to DD, | 21 |
| FRA88_CPS1CText | QKSRKVDSKSLFHYRQYSAGKAAKASTN* | AA Ct | 22 |
| fRA89_EPHB2 | REIQGIFFKEDSHKESNDCSCGG* | GG | 23 |
| FRA90_PDGFC | SLTDVALEHHEECDCVCRGSTGG* | GG | 24 |
| FRA91_ASCC3 | RRLDGKEEDEKMSRASDRFRGLR* | RG/R* dual | 25 |
| RA2102_degBon1 | TRGVEEVAEGVVLLRRRGN* | Rxx*/RxxG | 26 |
| RA2106_Clone1 | (GIPLR) NLGIR* | RG/R* dual | 27 |
| RA2107_Clone6 | (GIPLR) QRKLQRTSRG* | RG* | 28 |
| RA2108_Clone6GtoA | (GIPLR) QRKLQRTSRA* | RG* to A*, | 29 |
| RA2109_Clone8 | (GIPLR) PHKRLLKGSQYG* | RG *-like | 30 |

Further examples of C-degrons suitable for use in a fusion protein are listed below (see Bonger, Nat Chem Biol 7, 531-537):

| RA2103_ | TRGVEEVAEG | dual motif | SEQ ID NO: |
|---|---|---|---|
| degBon2 | VVLLRRRG* | (RG*) | 31 |
| RA2104_ | TRRRGN* | stronger | SEQ ID NO: |
| degBon3 | | variant | 32 |
| RA2105_ | \|RRRG* | strongest | SEQ ID NO: |
| degBon4 | | variant | 33 |

One example of an N-degron suitable for use in a fusion protein is listed below (see Bachmair et al, Cell 1989 56, 1019-1032). This sequence is a fusion of Ubiquitin and N terminus of B-gal.

| | | |
|---|---|---|
| Ubi-R | QIFVKTLTGKTITLEVESSDTIDN | SEQ ID |
| | VKSKIQDKEGIPPDQQRLIFAGKQ | NO: 34 |
| | LEDGRTLSDYNIQKESTLHLVLRL | |
| | RGGRHGSGAWLLPVSLVRRRTTLA | |
| | PNTQTASPRALADSLMQRS | |

One example of a PEST sequence suitable for use in a fusion protein is listed below (see Rogers et a; Science 1986 234: 364-8). There are many examples of PEST sequences.

| | | | SEQ ID |
|---|---|---|---|
| Name | Sequence | Origin | NO: |
| p53 | DDLLLPQDVEE | p53 | 35 |
| PEST | FFEGPSEALR | | |

Further examples of degrons that could be employed are shown below. These sequences are disclosed in Hon et al. (Nature 2002 417: 975-8), Fan et al. (Nat. Neurosci. 2014 17: 471-480), Gu et al. (Molecular and Cellular Biology 2000 20: 1243-1253), Melvin et al. (Analyst 2016 141:570-8) and Zhang et al. (Developmental Cell 2019 48: 329-344).

| | | | E3 | SEQ ID |
|---|---|---|---|---|
| Name | Sequence | Origin | Ligase | NO: |
| VHLdeg | ALAPYIP | HIF-1a | VHL | 36 |
| CMAdeg | KFERQKI | RNaseA- | Lysosome | 37 |
| | | hsc70- | | |
| | LDQRFFE | hemoglobin | | |
| MDM2deg | PLSSSVPS | p54 | MDM2 | 38 |
| | QKTYQGSY | (92-112) | | |
| | GFRLG | | | |
| MDM2(short) | GSYG | p54 | MDM2 | 39 |
| deg | | (92-112) | | |
| SPOP(2)deg | DVQKADVS | SRC3 | SPOP | 40 |
| | ST | | | |
| SPOP(3)deg | SPDSSTSP | Nanog | SPOP | 41 |
| BONGERdeg | RRRG | Synthetic | Unknown | 42 |
| iNOSdeg | DINNN | iNOS | Unknown | 43 |

In this fusion protein, the degron works in trans, meaning that the target protein that is degraded is a different protein, i.e., the protein that the fusion protein (which contains the degron) binds to.

### E3 Ligase Recruiting Domains

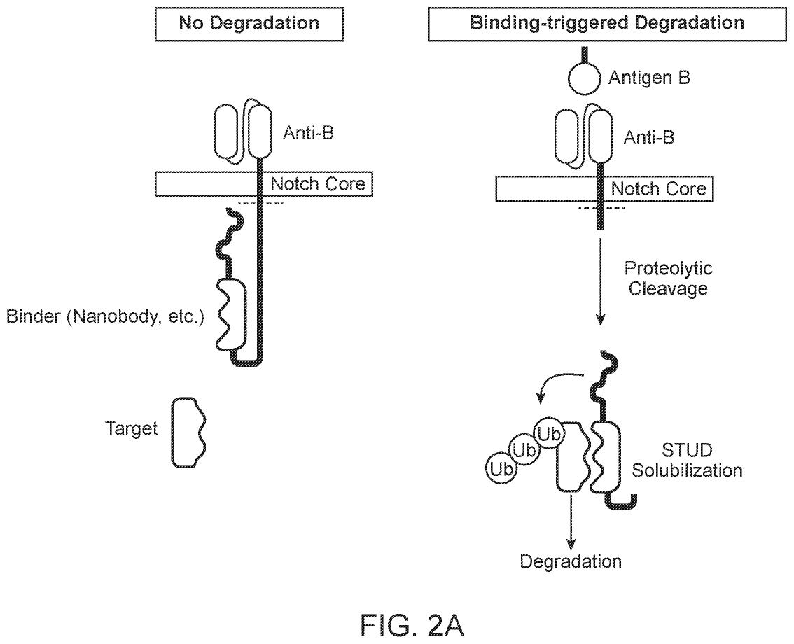

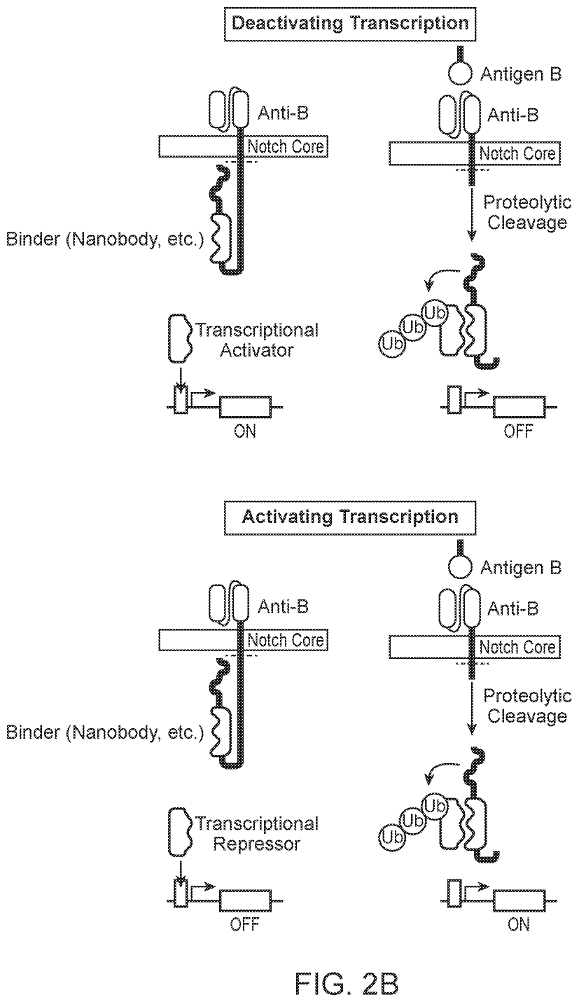

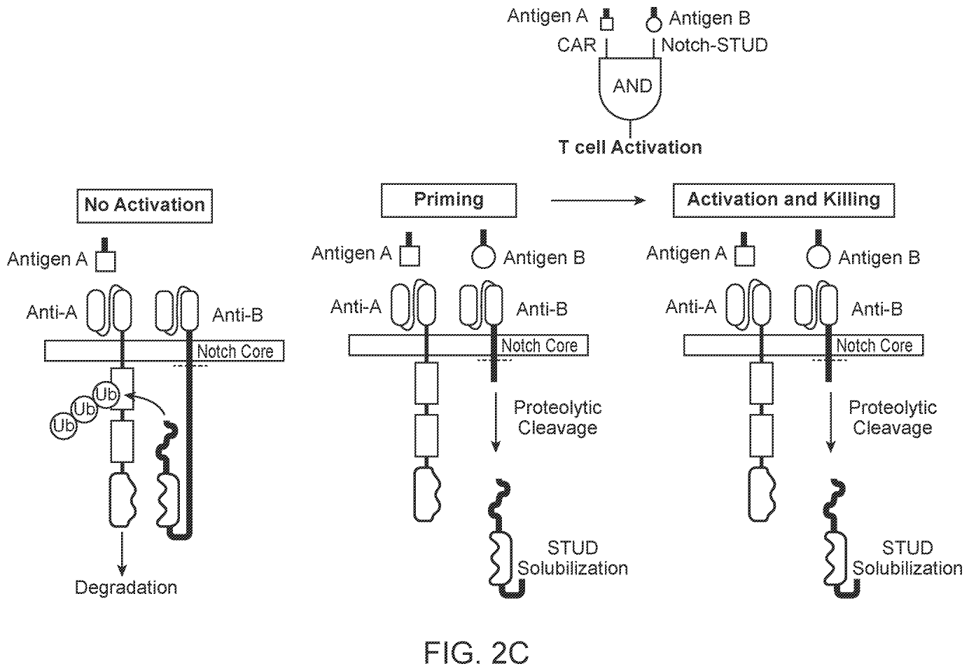

In the cell, the target-binding domain of the fusion protein binds to a target protein and recruits it into an E3-ligase complex, thereby causing the target to be ubiquitinated and degraded. In some embodiments, the E3 ligase recruiting domain of the fusion protein may interact with an E3 ligase directly or indirectly. In these embodiments, the E3 ligase is endogenous to the cell. FIG. 2 illustrates some of the current models of how substrates are recruited for degradation. As shown in panels A, B, D, E and F, many complexes contain an adapter protein (e.g., Skp1, Elongin B/C or DDB1) that links the E3 ligase (a cullin) to a protein that binds to the substrate. The protein that binds to the substrate is referred to as a “receptor” (and may be an F-box protein, VHL-box protein, DCAF, SOCS, for example). In one model (c), the receptor binds directly to the E3 ligase. The degradation domain of a fusion protein can contain any of the interaction domains shown in FIG. 2 (e.g., the E3 ligase interaction domain of an adapter protein or receptor, or the adapter protein-interaction domain of a receptor). As would be apparent, if the fusion protein contains the E3 ligase interaction domain of an adapter protein or receptor, or the adapter protein-interaction domain of a receptor, then the fusion protein does not need to contain other parts of the protein. For example, if the target binding domain of the fusion protein is from an adapter protein, then the fusion protein does not need to contain the part of the adapter protein that binds to the receptor. In these embodiments, the fusion protein may contain the E3 ligase binding domain of an adapter protein but not the receptor binding domain of the adapter protein. Likewise, if the target binding domain of the fusion protein is from a receptor protein, then the fusion protein does not need to contain the part of the receptor protein that binds to the endogenous substrate. In these embodiments, the fusion protein may contain the adapter protein binding domain of a receptor but not the substrate binding domain of the receptor.

In some embodiments, the E3 ligase recruiting domain can directly interact with a Cullin protein. Examples of E3 ligase recruiting domains that directly interact with a Cullin protein may be found in E3 complex adapter proteins and in some substrate receptors (e.g., BTB). These complexes promote the transfer of ubiquitin from the E2 to the substrate, which targets the protein for degradation. Many complexes contain an adapter protein (e.g., SKP1 for CUL1 and CUL7, Elongin B/C for CUL2 and CUL5, BTB for CUL3 and DDB1 for CUL4A/b) as well as a receptor protein (F-box proteins for CUL1, VHL-box proteins for CUL2, DCAFs for CUL4A and 4B, SOCS for CUL5 and Fbx W8 for CUL7) and a RING protein (RB1/2).

For example, an E3 ligase recruiting domain that directly interacts with an E3 ligase may have the Cullin binding region of an adapter protein, such as Skp1, ElonginB/C, or DDB1 (as illustrated in FIG. 2). These Cullin binding regions have been studied in depth (see, e.g., Schulman Nature 2000 408: 381-386, Zheng et al. Nature 2002 416: 703 and Fischer Nature 2014 512: 49-53) and the sequence of these domains can be readily derived from these studies. For example, Skp1 and ElonginC have a conserved BTB/POZ domain that interacts with CUL1 and CUL2/5, respectively.

In another example, an E3 ligase recruiting domain that directly interacts with a Cullin protein may have a BTB domain. Examples of BTB domains can be found in substrate receptors that interact directly with CUL3. Examples of such substrate receptors that directly interact with CUL3 include SPOP and KLHL family (e.g., Keap1) members. These Cullin binding regions have been studied in depth (see, e.g., Stogios et al. Genome Biology 2005 6: R82, Zhuang et al. Molecular Cell 2009 36: 39-50 and Lee et al. Molecular Cell 2009 36: 131-140) and the sequence of these domains can be readily derived from these studies.

In other embodiments, the E3 ligase recruiting domain may indirectly interact with an E3 ligase protein. This interaction may be via an adapter protein. Examples of E3 ligase recruiting domains that indirectly interact with an E3 ligase may be found in some E3 substrate receptors (e.g., those receptors that interact with a Cullin via an adapter protein).

For example, an E3 ligase recruiting domain that indirectly interacts with an E3 ligase may have an F-box. Examples of F-box domains can be found in E3 substrate receptors that interact with Cullin-1 or Cullin-7 via Skp1. Canonical F-box proteins that bind Skp1 include FBW1A (beta-TRCP), Skp2, and Fbw7. The F box has been studied in depth (Su et al. Proc. Natl. Acad. Sci. 2003 100: 12729-12734; Schulman, Nature 2000 408:381-386, Yumimoto Journal of Biological Chemistry 2-13 288: 28488-28502 and Skaar, Nature Reviews Molecular Cell Biology 2013 14: 369-381) and the sequence of this domain can be readily derived from these studies.

In another example, an E3 ligase recruiting domain may have a VHL- or SOCS-box. Examples of VHL- and SOCS-box domains can be found in E3 substrate receptors that interact Cullin-2 or Cullin-7 via Elongin B/C. Examples of F-box domains include members of suppressors of cytokine signaling (SOCS) family of proteins (e.g., Socs1, Socs3) as well as pVHL. The structure of these domains has been studied in depth (see. e.g., Liau et al. Nature Comm 2018 9: 1558, Stebbins et al. Science 1999 284: 455-461, Kamura, Genes & Development 2003 18: 3055-3065 and Linossi IUBMB Life 2012 64: 316-323) and the sequence of this domain can be readily derived from these studies.

In another example, an E3 ligase recruiting domain may have a WDXR motif. Examples of WDXR motifs can be found in E3 substrate receptors that interact with Cullin-4A or 4B, via DDB1. Examples of WDXR motifs include those of the DCAF family of proteins (e.g., DCAF1, DCAF9 and DDB2). DDB1 interacts with CUL4 (similar to Skp1), and proteins such as DCAF1 provide the substrate recognition (similar to Skp2). DCAF1-type proteins use repeats of WD40 motifs, in which WDXR motifs are embedded, to bind to DDB1. The interactions between DDB11/WDXR proteins and E3 ligases have been studied in depth (see. e.g., Scrima et al. Cell 2008 135: 1213-1223, Yumimoto et al Journal of Biological Chemistry 2013 288: 28488-28502, Fischer et al Cell 2011 147: 1024-39, Fischer Nature 2014 512: 49-53, Schabla Journal of Molecular Cell Biology 2019 11: 725-735 and Jackson et al. Trends Biochem Sci. 2009 34: 562-570) and the sequence of this domain can be readily derived from these studies.

In alternative embodiments, the fusion protein could be a fusion between a target binding domain and an E3 ligase, such as one of the Cullins or E3 ubiquitin-protein ligase CHIP (see, e.g., Portnoff et al. J. Biol. Chem. 214 289: 7844 7855).

Finally, it may be possible to directly link the domain from Rbx1 that binds to E2 to a target binding domain. This fusion may still bind to the E3 ligase (as shown in FIG. 2) or it may bypass the E3 ligase if E2 can transfer ubiquitin onto substrates autonomously.

In any embodiment, the degradation domain, the target-binding domain and/or the linker may be selected or modified so that there are no lysines on the surface of the domain, thereby protecting the fusion protein from cis-ubiquitination and subsequent auto-degradation. In these embodiments, this domain may be designed by analyzing a sequence through a structural prediction program, identifying lysines on the surface of a domain, and then changing the lysines to another residue (e.g., arginine, which is similar to lysine but not targeted by the ubiquitin ligase). In some embodiments, all of the lysines in one or more of the domains of the fusion protein may be modified to be arginines. In these embodiments, the fusion protein may be lysine free. In other embodiments, a subset of lysines (e.g., 1, 2, 3, 4, 5, 6 or 7 lysines) may be mutated to tune the balance of cis-versus trans-ubiquitination. These lysines may be identified based on their propensity for ubiquitination or surface accessibility. This strategy may be useful for tuning the activity of the protein degrader tool.

### Force Sensing Regions and Force-Dependent Cleavage Sites

As noted above, the fusion protein cleaves to release the intracellular domain when the extracellular domain of the fusion protein engages with a cognate antigen on another cell. As such, in many cases, the fusion protein will contain a force sensing region (which is typically in the extracellular domain) and one or more force-dependent cleavage sites that are cleaved when the force sensing region is activated. The position of the force-dependent cleavage sites may vary and, in some embodiments the fusion protein may contain at least two cleavage sites. In some cases, one of the cleavage sites may be extracellular and the other may be in the transmembrane domain or within 10 amino acids of the transmembrane domain in the intracellular domain. In any embodiment, the force sensing region and/or the one or more force-dependent cleavage sites may be from a Delta/Serrate/Lag2 (DSL) superfamily protein, as reviewed by Pintar et al (Biology Direct 2007 2: 1-13). For example, the force sensing region and/or the one or more force-dependent cleavage sites may be from Notch (see Morsut Cell. 2016 164: 780-91), von Willebrand Factor (vWF), amyloid-beta, CD16, CD44, Delta, a cadherin, an ephrin-type receptor or ephrin ligand, a protocadherin, a filamin, a synthetic E cadherin, interleukin-1 receptor type 2 (IL1R2), major prion protein (PrP), a neuregulin or an adhesion-GPCR. Several other examples of this type of protein are known and listed in Pintar, supra. Many members of this family appear to share a similar architecture a region that unfolds and opens up a protease cleavage site (e.g., EGF-like repeats; see Cordle et al Nat. Struct. Mol. Biol. 2008 15: 849-857), a trans-membrane segment, and a relatively short (˜100-150 amino acids) intracellular domain. These sequences permit the binding-triggered release of a transcriptional regulator from the membrane in their natural environment and can be readily adapted herein, where the term transcription regulator refers to polypeptides that have both a DNA binding domain and a transcriptional activation domain, or polypeptide that has one or the other, where the other is already in the cell and the polypeptides are designed to dimerize with one another, i.e., in the form of a “split” transcription factor.

In some cases, the one or more ligand-inducible proteolytic cleavage sites are selected from S1, S2, and S3 proteolytic cleavage sites. In some cases, the S1 proteolytic cleavage site is a furin-like protease cleavage site comprising the amino acid sequence Arg-X-(Arg/Lys)-Arg, where X is any amino acid. In some cases, the S2 proteolytic cleavage site ADAM-17-type protease cleavage site comprising an Ala-Val dipeptide sequence. In some cases, the S3 proteolytic cleavage site is a γ-secretase cleavage site comprising a Gly-Val dipeptide sequence. The S3 proteolytic cleavage site is in the transmembrane domain. In many cases, the shear force generated by binding of the extracellular domain of this fusion protein to another cells unfolds the force sensing region (which, in the case of Notch contains EGF-like repeats whereas in other protein is made up of other sequences such as the A2 domain in vWF (see, e.g., J Thromb Haemost. 2009 7:2096-105, Lippok Biophys J. 2016 110: 545-54, Lynch Blood. 2014 123: 2585-92, Crawley, Blood. 2011 118:3212-21 and Xy J Biol Chem. 2013 288:6317-24) or modified A2 domain that has, e.g., the R1597W, E1638K and I1628T substitutions. The architecture of such proteins is described in, e.g., Morsut Cell. 2016 164: 780-91, WO2016138034 and WO2019099689, among other places).

In some cases, the Notch receptor polypeptide includes an S1 ligand-inducible proteolytic cleavage site. An S1 ligand-inducible proteolytic cleavage site can be located between the HD-N segment and the HD-C segment. In some cases, the S1 ligand-inducible proteolytic cleavage site is a furin-like protease cleavage site. A furin-like protease cleavage site can have the canonical sequence Arg-X-(Arg/Lys)-Arg, where X is any amino acid; the protease cleaves immediately C-terminal to the canonical sequence. For example, in some cases, an amino acid sequence comprising an S1 ligand-inducible proteolytic cleavage site can have the amino acid sequence GRRRRELDPM (SEQ ID NO: 44), where cleavage occurs between the “RE” sequence. As another example, an amino acid sequence comprising an S1 ligand-inducible proteolytic cleavage site can have the amino acid sequence RQRRELDPM (SEQ ID NO: 45), where cleavage occurs between the “RE” sequence.

In some cases, the Notch receptor polypeptide includes an S2 ligand-inducible proteolytic cleavage site. An S2 ligand-inducible proteolytic cleavage site can be located within the HD-C segment. In some cases, the S2 ligand-inducible proteolytic cleavage site is an ADAM-17-type protease cleavage site. An ADAM-17-type protease cleavage site can comprise an Ala-Val dipeptide sequence, where the enzyme cleaves between the Ala and the Val. For example, in some cases, amino acid sequence comprising an S2 ligand-inducible proteolytic cleavage site can have the amino acid sequence KIEAVKSE (SEQ ID NO: 46), where cleavage occurs between the “AV” sequence. As another example, an amino acid sequence comprising an S2 ligand-inducible proteolytic cleavage site can have the amino acid sequence KIEAVQSE (SEQ ID NO: 47), where cleavage occurs between the “AV” sequence. This ADAM protease is not essential for force sensing switches to work, however, Zhu et al (BioRxiv 2021 2021.05.21.445218).

In some cases, the Notch receptor polypeptide includes an S3 ligand-inducible proteolytic cleavage site. An S3 ligand-inducible proteolytic cleavage site can be located within the TM domain. In some cases, the S3 ligand-inducible proteolytic cleavage site is a gamma-secretase (γ-secretase) cleavage site. A γ-secretase cleavage site can comprise a Gly-Val dipeptide sequence, where the enzyme cleaves between the Gly and the Val. For example, in some cases, an S3 ligand-inducible proteolytic cleavage site has the amino acid sequence VGCGVLLS (SEQ ID NO: 48), where cleavage occurs between the “GV” sequence. In some cases, an S3 ligand-inducible proteolytic cleavage site comprises the amino acid sequence GCGVLLS (SEQ ID NO: 49).

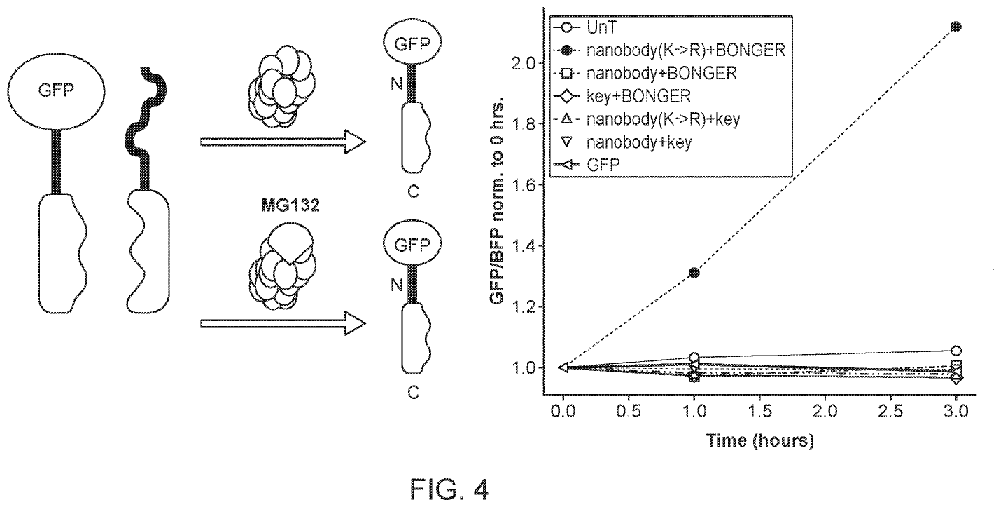

In some cases, the Notch receptor polypeptide lacks an S1 ligand-inducible proteolytic cleavage site. In some cases, the Notch receptor polypeptide lacks an S2 ligand-inducible proteolytic cleavage site. In some cases, the Notch receptor polypeptide lacks an S3 ligand-inducible proteolytic cleavage site. In some cases, the Notch receptor polypeptide lacks both an S1 ligand-inducible proteolytic cleavage site and an S2 ligand-inducible proteolytic cleavage site. In some cases, the Notch receptor polypeptide includes an S3 ligand-inducible proteolytic cleavage site; and lacks both an S1 ligand-inducible proteolytic cleavage site and an S2 ligand-inducible proteolytic cleavage site. Examples are depicted schematically in FIG. 4G.

In other embodiments, the fusion protein may have an vWF A2 sequence or a variation thereof, an ADAMTS13 cleavage site (which may be described by the consensus sequence HEXXHXXGXXHD SEQ ID NO: 50; Crawley, Blood. 2011 118:3212-21), and an S3 or γ-secretase cleavage site, although many other arrangements exist.

In some embodiments, the fusion protein may be a SNIPR, as described in Zhu et al BioRxiv 2021 2021.05.21.445218).

### Target Proteins

The target protein can be endogenous (i.e., native) to the cell or exogenous to the cell (i.e., expressed using recombinant means). In embodiments in which the target protein is exogenous to the cell, the target protein may be engineered to contain a binding site for the second binding domain of the fusion protein, if necessary.

In some embodiments, the target protein may be localized to the plasma membrane. For example, the target protein may be a transmembrane protein (e.g., a recombinant receptor such as a chimeric TCR, CAR, iCAR or synNotch protein, or an endogenous receptor such as an immune checkpoint selected from PD1, CTLA4, BTLA, CD160, KRLG-1, 2B4, Lag-3, Tim-3, TIGIT, etc.). or the target protein may be directly or indirectly associated with a transmembrane protein (e.g., a protein that binds to a transmembrane protein, such as PI3K, GRB2, TRAF1, ZAP70, TRAF6, IRAK1, IRAK4, MyD88, TIRAP, TRAM, TRIF, TBK1, a membrane-associated tyrosine kinase such as Lck, or any other component of the T cell signaling pathway such as LAT or Ras, etc. In particular embodiments, the target protein may be an immune receptor, e.g., a CAR or chimeric costimulatory receptor (CCR).

In other embodiments, the target protein is not localized to the plasma membrane. In these embodiments, the target protein may be cytoplasmic, nuclear, or localized in the endoplasmic reticulum, mitochondria or lysosome, etc. For example, if the target protein is cytoplasmic, it may be a kinase, phosphatase, or an enzyme involved in the synthesis of a secondary messenger, e.g., adenylyl cyclase, which catalyzes the production of cAMP. If the target protein is nuclear, the target protein may be a DNA binding protein such as a transcription factor or a chromatin modifier such as a histone methylase or demethylase. Of particular interest are transcription factors that are implicated in cell fate and differentiation. A comprehensive list of transcription factors that are involved in human cell fate is provided in Ng et al (Nature Biotechnology 2020 PMID: 33257861) and several strategies for engineering cell fates using transcription factors is described in Koch (Nature Reviews Genetics 2021 22: 68). Any of these transcription factors could be regulated using the present system. For example, the target protein could a member of the Oct, Sox Nanog or Nestin families (e.g., Oct4, Sox2 etc) (which are involved in pluripotency), NFAT, AP-1, FOXP3, PU1 or GATA3 (which are important for T cell fate), or members of the bZIP family (Jun, Fos, etc.), TOX, or NR4A (which are involved in T cell exhaustion and related processes).

In some embodiments, the transcription factor may be a transcriptional activator or a transcriptional repressor. Targeting a transcriptional activator may decrease the expression of a target gene and targeting a transcriptional repressor may increase the expression of a target gene. As would be apparent, the target transcription factor may be endogenous to the cell or it could be engineered. In some embodiments, the promoter acted upon by the transcription factor may be endogenous to the cell or recombinant, i.e., part of an expression cassette for the expression of a recombinant protein (non-natural protein), where the promoter and coding sequence are not found together in nature.

As noted above, in some embodiments, the target protein is a transmembrane protein. In these embodiments, the target protein may comprise: i. an extracellular binding domain comprising a second binding moiety that is capable of specifically binding to a second cell surface marker; ii. a transmembrane domain; and iii. comprises an effector region (e.g., a costimulatory domain, ITAM or ITIM domain) that is activated by binding of the extracellular binding domain to a target via the first binding region. Examples of such target proteins included chimeric antigen receptors (CARs), iCARs, synNotch receptors, chimeric T cell receptors, etc.

In some embodiments, the target protein may be a therapeutic protein that, when expressed on the surface of an immune cell, activates the immune cell or inhibits activation of the immune cell when it binds to an antigen on the diseased cell. In these embodiments, the therapeutic protein may be a chimeric antigen receptor (CAR) or a T cell receptor (TCR). In these embodiments, the cell may be a T cell that expresses a CAR or TCR, where the CAR or TCR comprises an extracellular domain, a transmembrane region and an intracellular signaling domain; where the extracellular domain comprises a ligand or a receptor and the intracellular signaling domain comprises an ITAM domain, e.g., the signaling domain from the zeta chain of the human CD3 complex (CD3zeta), and, optionally, one or more costimulatory signaling domains, such as those from CD28, 4-1BB and OX-40. The extracellular domain contains a recognition element (e.g., an antibody or other target-binding scaffold) that enables the CAR to bind a target. In some cases, a CAR comprises the antigen binding domains of an antibody (e.g., an scFv) linked to T-cell signaling domains. In some cases, when expressed on the surface of a T cell, the CAR can direct T cell activity to those cells expressing a receptor or ligand for which this recognition element is specific. As an example, a CAR that contains an extracellular domain that contains a recognition element specific for a tumor antigen can direct T cell activity to tumor cells that bear the tumor antigen. The intracellular region enables the cell (e.g., a T cell) to receive costimulatory signals. The costimulatory signaling domains can be selected from CD28, 4-1BB, OX-40 or any combination of these. Exemplary CARs comprise a human CD4 transmembrane region, a human IgG4 Fc and a receptor or ligand that is tumor-specific, such as an IL13 or IL3 molecule. In these embodiments, activation of a CAR activates the immune cell.

Alternatively, the therapeutic protein may be an inhibitory immune cell receptor (iICR) such as an inhibitory chimeric antigen receptor (iCAR), wherein binding of the iICR to a marker on another cell inhibits activation of the immune cell on which the iICR is expressed. Such iICR proteins are described in e.g., WO2017087723, Fedorov et al. (Sci. Transl. Med. 2013 5: 215ra17) and other references cited above, which are incorporated by reference for that description and examples of the same. In some embodiments, an inhibitory immunoreceptor may comprise an intracellular immunoreceptor tyrosine-based inhibition motif (ITIM), an immunoreceptor tyrosine-based switch motif (ITSM), an NpxY motif, or a YXXΦ motif. Exemplary intracellular domains for such molecules may be found in PD1, CTLA4, BTLA, CD160, KRLG-1, 2B4, Lag-3, Tim-3 and other immune checkpoints, for example. See, e.g., Odorizzi and Wherry (2012) J. Immunol. 188:2957; and Baitsch et al. (2012) PLoSOne 7: e30852.

If the target protein is endogenous, then the fusion protein may contain a domain of a natural binding partner of the target protein, or another specific binding domain such as a nanobody or scFv.

If the target protein is exogenous, then in some cases the target protein can be engineered to contain a binding site for the dimerization domain of the fusion protein. In these embodiments, the target protein can be designed to contain an epitope tag (e.g., a hemagglutinin, FLAG, c-myc, ALFA, or V5 tag), and the like to which the dimerization domain binds. Alternatively, the target protein can be designed to contain a synthetic leucine zipper domain or any of the other domains described above, that heterodimerizes with a complementary synthetic leucine zipper domain in the fusion protein, as discussed above.

In some cases, binding of the fusion protein to the target protein may be conditional. In these embodiments, target binding domain of the fusion protein and the target protein may be engineered to only bind to one another in the presence of a dimerization agent. Examples of pairs of protein domains that conditionally dimerize with one another include: FKBP and FKBP (which dimerize in the presence of rapamycin), FKBP and CnA (which dimerize in the presence of rapamycin), FKBP and cyclophilin (which dimerize in the presence of rapamycin), FKBP and FRG (which dimerize in the presence of rapamycin), GyrB and GyrB (which dimerize in the presence of coumermycin), DHFR and DHFR (which dimerize in the presence of methotrexate), DmrB and DmrB (which dimerize in the presence of AP20187), PYL and ABI (which dimerize in the presence of abscisic acid), Cry2 and CIB1 (which dimerize in the presence of blue light); GAI and GID1 (which dimerize in the presence of gibberellin) and a ligand-binding domain of a nuclear hormone receptor, and a co-regulator of the nuclear hormone receptor (which dimerize in the presence of a nuclear hormone, agonists thereof and antagonists thereof, e.g., tamoxifen). In embodiments in which rapamycin can serve a dimerizer, a rapamycin derivative or analog can also be used.

In any embodiment, expression of the fusion protein may be inducible, tissue-specific, or constitutive. This may be done by operably linking the coding sequence for the fusion protein to an appropriate promoter.

### Cells

A therapeutic cell (e.g., a recombinant immune cell such as a CAR T, a Treg cell or stem cell) that expresses a fusion protein (i.e., contains an expression cassette comprising a promoter and, operably linked to the promoter, a coding sequence that encodes the fusion protein described above) is also provided. The therapeutic cell may be genetically modified to contain a nucleic acid comprising an expression cassette comprising a promoter and a coding sequence for the fusion protein as described above. As noted above, the coding sequence may additionally encode a signal sequence, thereby targeting the protein to the plasma membrane.

In some instances, a therapeutic cell is an immune cell. Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or a progenitor thereof, obtained from an individual. As another example, the cell is a cytotoxic cell, or a progenitor thereof, obtained from an individual. As another example, the cell is a stem cell or progenitor cell obtained from an individual.

In some cases, the cell is an immune cell, e.g., a T cell, a B cell, a macrophage, a dendritic cell, a natural killer cell, a monocyte, etc. In some cases, the cell is a T cell. In some cases, the cell is a cytotoxic T cell (e.g., a CD8+ T cell). In some cases, the cell is a helper T cell (e.g., a CD4+ T cell). In some cases, the cell is a regulatory T cell (“Treg”). In some cases, the cell is a B cell. In some cases, the cell is a macrophage. In some cases, the cell is a dendritic cell. In some cases, the cell is a peripheral blood mononuclear cell. In some cases, the cell is a monocyte. In some cases, the cell is a natural killer (NK) cell. In some cases, the cell is a CD4+, FOXP3+ Treg cell. In some cases, the cell is a CD4+, FOXP3-Treg cell. The immune cell can be immunostimulatory or immunoinhibitory.

In some embodiments, the therapeutic cell may be a CAR T cell.

Suitable therapeutic cells also include stem cells, progenitor cells, as well as partially and fully differentiated cells. Suitable cells include neurons; liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); and a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs).

Cells of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo. For example, in some embodiments, nucleic acid encoding the current proteins (e.g., mRNA) can be delivered in vivo, e.g., using T cell-targeted lipid nanoparticles (LNPs).

As would be apparent, the cell may further comprise the target protein (i.e., a protein to which the fusion protein dimerizes and induces degradation of). In these embodiment the target protein, may be localized at the plasma membrane (either because it has a transmembrane sequence itself or it binds to a transmembrane protein) and comprises a target dimerization domain to which the first dimerization domain of (c)(i) binds. In these embodiments, binding of the fusion protein to the target protein via the first and target dimerization domains induces proteosome-mediated degradation of the target protein.

In some instances, the cell is obtained from an individual. For example, in some cases, the cell is a primary cell. As another example, the cell is a stem cell or progenitor cell obtained from an individual.

As one non-limiting example, in some cases, the cell is an immune cell obtained from an individual. As an example, the cell can be a T lymphocyte obtained from an individual. As another example, the cell is a cytotoxic cell (e.g., a cytotoxic T cell) obtained from an individual. As another example, the cell can be a helper T cell obtained from an individual. As another example, the cell can be a regulatory T cell obtained from an individual. As another example, the cell can be an NK cell obtained from an individual. As another example, the cell can be a macrophage obtained from an individual. As another example, the cell can be a dendritic cell obtained from an individual. As another example, the cell can be a B cell obtained from an individual. As another example, the cell can be a peripheral blood mononuclear cell obtained from an individual.

In some cases, the host cell is not an immune cell. In these embodiments, the host cell may be a somatic cell, e.g. a fibroblast, a hematopoietic cell, a neuron, a pancreatic cell, a muscle cell, a bone cell, a hepatocyte, a pancreatic cell, an epithelial cell, an endothelial cell, a cardiomyocyte, a T cell, a B cell, an osteocyte, or a stem cell, and the like.

As noted above, the rate at which the target protein is degraded may increase when the extracellular domain of the fusion protein and extracellular domain of the target transmembrane protein are bound to markers on the same cell. In these embodiments, the first dimerization domain and the target dimerization domain may bind to one another with a low affinity, as discussed above.

### Protein Circuits

Protein circuits are also provided, where a protein circuit may comprise a fusion protein as described above and the target protein. In these circuits, binding of the first binding moiety of the fusion protein to the cell surface protein on another cell releases the intracellular domain and alters degradation of the target protein. For example, if the target protein is localized to the plasma membrane then the binding event should result in a decrease in degradation of the target protein. Conversely, if the target protein is not localized to the plasma membrane then the binding event should result in an increase in degradation of the target protein.

If the fusion protein targets an immunoreceptor such as a CAR or a binding-triggered transcription switch (e.g., a synNotch) that induces expression of a CAR, the resulting protein circuits can provide a “AND” gate, i.e., provide a way of inducing signaling within a cell in response only if there are two or more distinct ligands on another cell (see e.g., Roybal et al Cell 2016 164: 770-9, Ebert et al Biochem Soc Trans. 2018 46: 391-401 and WO2005010198). “OR” circuits can be readily configured by targeting factors, or other components of a signal transduction pathway.

In some embodiments, the protein circuit may comprise i. a fusion protein as described above; and ii. a target protein comprising: an extracellular binding domain that is capable of specifically binding to a cell surface marker (e.g., a cancer marker), a transmembrane domain; and an intracellular domain that comprises a domain to which the second dimerization domain of the fusion protein binds. In other embodiments noted above, the target protein may be a cellular protein or transcription factor.

If the target protein is an immunoreceptor, then fusion protein and the target protein (which is a plasma membrane protein) both specifically bind to proteins that are on the surface of other cells. In these embodiments, the circuit results in a decrease in degradation of the target protein in the cell when the cell comes in contact with another cell that has the both the first and second proteins (i.e., the protein bound by the fusion protein and the protein bound by the immunoreceptor) on its surface. As such, the present circuit provides a way for therapeutic immune cells (e.g., chimeric antigen receptor (CAR) T cells) to discriminate between tumor and normal tissues in the treatment of cancer.

In some embodiments, the fusion protein (and the target protein, if the target protein is an immunoreceptor) may bind to a cancer marker, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin ανβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin, for example. Combinations of markers that could be used in the present circuits are described in, e.g., Dannenfelser et al (Cell Syst 2020 11: 215-228).

### Nucleic Acids

Given that the genetic code is known, a sequence that encodes the fusion protein can be readily determined. In some embodiments, the coding sequence may be codon optimized for expression in mammalian (e.g., human or mouse) cells, strategies for which are well known (see, e.g., Mauro et al., Trends Mol. Med. 2014 20: 604-613 and Bell et al Human Gene Therapy Methods 27: 6). As would be understood, the coding sequence may be operably linked to a promoter, which may be inducible, tissue-specific, or constitutive. In some embodiments, the promoter may be activated by an engineered transcription factor that is heterologous to the cell, e.g., a Gal4-, LexA-, Tet-, Lac-, dCas9-, zinc-finger- and TALE-based transcription factors.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used. NK cell-specific expression can be achieved by use of an Ncr1 (p 46) promoter; see, e.g., Eckelhart et al. (2011) Blood 117:1565.

In some cases, the promoter is a cardiomyocyte-specific promoter. In some cases, the promoter is a smooth muscle cell-specific promoter. In some cases, the promoter is a neuron-specific promoter. In some cases, the promoter is an adipocyte-specific promoter. Other cell type-specific promoters are known in the art and are suitable for use herein.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., Hum Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

### Methods and Uses

Also provided herein is a method for modulating degradation of a target protein in the cell described above. In some embodiments, this method may comprise introducing a first cell to a second cell, wherein the first cell comprises a fusion protein as described above; and the target protein, and the second cell comprises the cell surface protein on its surface, wherein binding of the first cell to the second cell via the fusion protein releases the intracellular domain, thereby altering degradation of the target protein. As noted above, if the target protein is localized to the plasma membrane then the introducing step results in a decrease in degradation of the target protein. If target protein is not localized to the plasma membrane then introducing step results in an increase in degradation of the target protein.

The introducing can be done in vitro (using isolated cells), ex vivo (using cells that have been taken from a person) or in vivo. In the latter case, the introducing may be done by administering the first cell to a subject, e.g., by injection.

In some embodiments, the method may be used to manipulate cell differentiation.

In some embodiments, the cell may be used in a method of treatment that comprises administering the cell to a patient in need thereof.

In some embodiments, the patient may have cancer, e.g., breast cancer, B cell lymphoma, pancreatic cancer, Hodgkin lymphoma cell, ovarian cancer cell, prostate cancer, mesothelioma, lung cancer (e.g., a small cell lung cancer), non-Hodgkin B-cell lymphoma (B-NHL) cell, ovarian cancer, a prostate cancer, melanoma cell, a chronic lymphocytic leukemia cell, acute lymphocytic leukemia cell, a neuroblastoma, a glioma, a glioblastoma, a medulloblastoma, a colorectal cancer, etc. In these embodiments, the therapeutic cell may be a CAR T cell that comprises a CAR that recognizes an antigen expressed by the cancer cells.

In some embodiments, the patient may have an inflammatory condition or autoimmune disease. In these embodiments, the cell may be a T-helper cell or a Treg for use in an immunomodulatory method. Immunomodulatory methods include, e.g., enhancing an immune response in a mammalian subject toward a pathogen; enhancing an immune response in a subject who is immunocompromised; reducing an inflammatory response; reducing an immune response in a mammalian subject to an autoantigen, e.g., to treat an autoimmune disease; and reducing an immune response in a mammalian subject to a transplanted organ or tissue, to reduce organ or tissue rejection.

In one exemplary embodiment, the fusion protein may be used to degrade transcription facts that positively regulate T-cell exhaustion, thereby providing a way to decrease or eliminate T cell exhaustion in an antigen-specific manner.

In some embodiments, the patient is in need of a stem cell transplantation.

### EXAMPLES

The following examples are put forth so as to provide those of ordinary skill in the art with a complete disclosure and description of how to make and use the present invention, and are not intended to limit the scope of what the inventors regard as their invention.

### Example 1

### Cis-Ubiquitination can be Prevented by Substituting the Lysines in a STUD

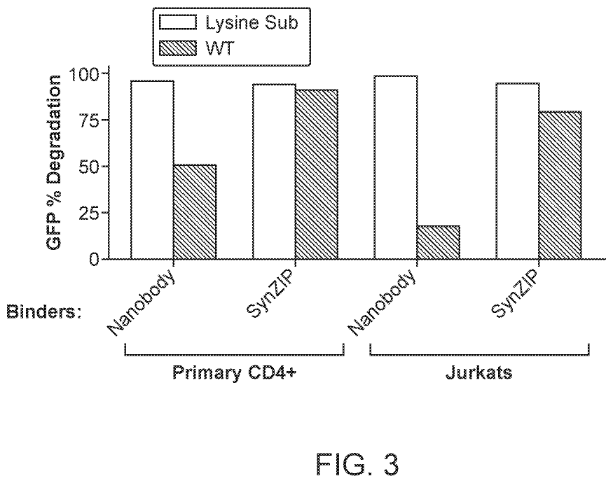

This protein degradation tool has the potential to ubiquitinate target lysines on both the target of interest (trans-ubiquitination), as well as on the tool itself (cis-ubiquitination). cis-ubiquitination may limit the effectiveness of the STUD by degrading the STUD before it has the chance to interact with its target. To solve this problem, the lysines on the protein targeting domain of the STUD were mutated to arginines (K->R), thus preventing cis-ubiquitination². An assay was developed to test the functionality of a STUD by measuring degradation of a cytosolic GFP. The GFP was targeted for degradation using either a GFP nanobody or a SynZIP17 that was fused to the GFP. The target GFP was transduced into either Jurkat cells or primary human T cells using lentivirus and the STUD was introduced via a second lentivirus. It was observed that the lysine substitution significantly improved the activity of the GFP nanobody STUD, whereas the mutation only moderately improved the activity of the SynZIP STUD. These results are shown in FIG. 3. This trend was consistent between primary human CD4+ T cells and Jurkats. Given these results, it should be possible to use the number of lysines on the STUD as a strategy for tuning the activity of the STUD, where more mutated lysines increases the activity of the STUD.

### Example 2

### STUD-Induced Degradation is Mediated Via the Proteasome

The mechanism of how the STUD reduces GFP was explored. Primary human CD4+ T cells expressing the GFP nanobody STUD were fed with the MG132 proteasome inhibitor and the change in fluorescence was measured over time. These results are shown in FIG. 4. Cells expressing a functional STUD should display an increase in fluorescence over time as the proteasome inhibitor took effect. After three hours of exposure to the drug, it was observed that only the cells expressing the functional nanobody STUD (nanobody(K−>R)+Bonger) displayed an increase in GFP fluorescence. This indicates that the observed reduction in GFP is mediated by degradation via the proteasome rather than a mechanism associated with the protein-protein interaction alone.

### Example 3

### STUD Activity can be Optimized Using a Linker

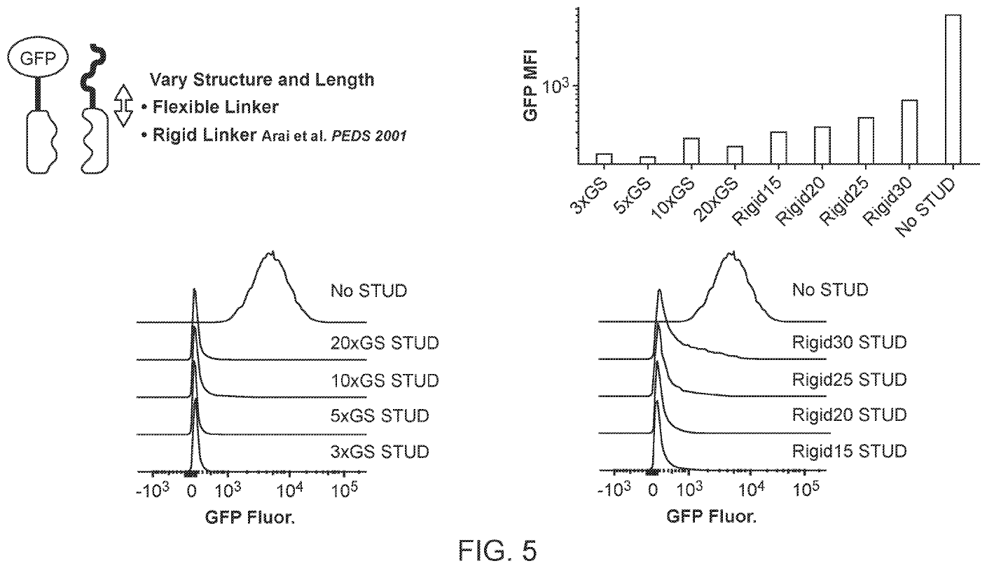

The STUD was optimized by screening multiple lengths of two different classes of linkers. In these constructs, the linker was added between a SynZIP protein binding domain and the Bonger degron. It was hypothesized that a flexible Gly-Ser linker may facilitate target degradation by increasing the accessibility of the E3 ligase to reach target lysine residues on the surface of the target protein, whereas a rigid helical linker may increase the distance between the E3 ligase and target lysines and reduce degradation. These experiments used the SynZIP STUD that targets cytosolic GFP-SZ17 as described above. Four lengths of linker for both the flexible and rigid linker. The flexible linker generally performed better than the rigid linker, with little variation in degradation efficiency observed within the different flexible linker lengths (FIG. 5). However among the flexible linkers the 5xGS performed the best. This STUD (with the SynZIP(K->R), optimized linker and C-terminal RRRG (SEQ ID NO: 51), or SynZIP18(K->R)-5xGS-RRRG (SEQ ID NO: 52)) is referred to as the “soluble stud” and used in the following experiments.

### Example 4

### Transcription Factors can be Targeted

Lysine substitution and linker length/type optimization served as a framework for optimizing future STUD iterations that use other protein targeting domains and/or degradation domains, e.g., degrons. Depending on the application, different synthetic protein targeting domains may be more suitable, and it is also possible to utilize endogenous protein targeting domains that bind to or interact with an endogenous protein without the need for modification of the endogenous protein. Furthermore, different degrons may be utilized to vary the conditions under which the STUD is active, or confine the activity of the STUD to different compartments of the cell where the degron is active.

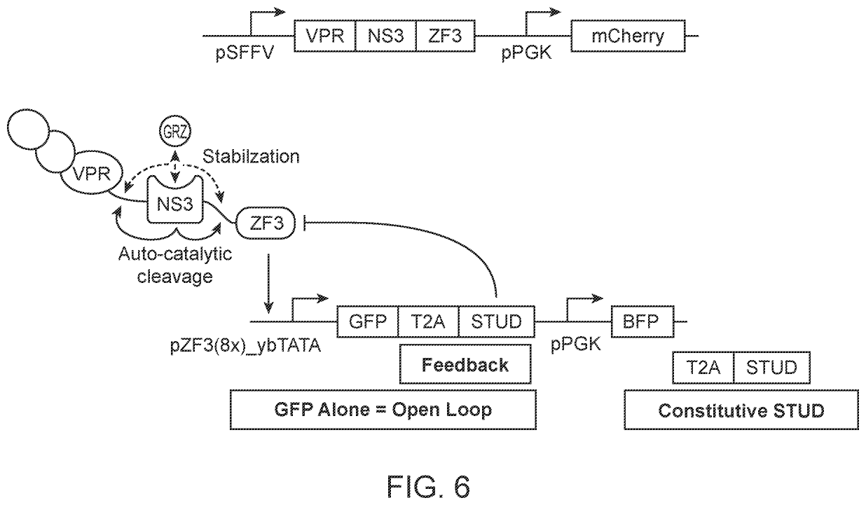

A transcription factor was targeted for degradation using the soluble STUD described above. Modulating a transcription factor allows one to affect the output of a functional protein. These experiments were done using a previously developed grazoprevir (GRZ) drug-inducible zinc-finger transcription factor system (VPR-NS3-ZF3). To induce degradation of this transcription factor SynZIP17 to the C-terminus of this protein. Degradation of the TF was measured by observing changes in GFP reporter output driven by the pZF3(8x)ybTATA promoter. Two different methods were used for STUD expression: constitutive STUD expression, or inducible STUD expression, which should drive negative feedback in the system (FIG. 6).

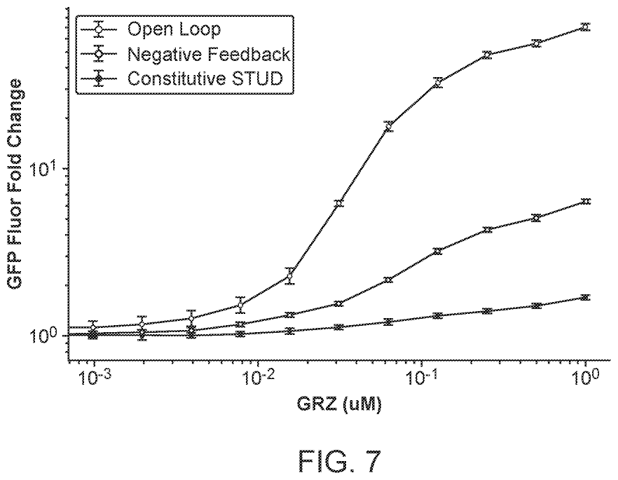

The dose responses of the three circuit variants were compared to assess the functionality of the STUD. It was found that constitutive expression of the STUD abolished nearly all output from the pZF3, whereas feedback expression of the STUD generated an intermediate dose response (FIG. 7). This demonstrates that the soluble STUD can not only degrade functional proteins in the cell, but also be used as a powerful tool for building genetic circuits.

### Example 5

### Transmembrane Proteins can be Targeted

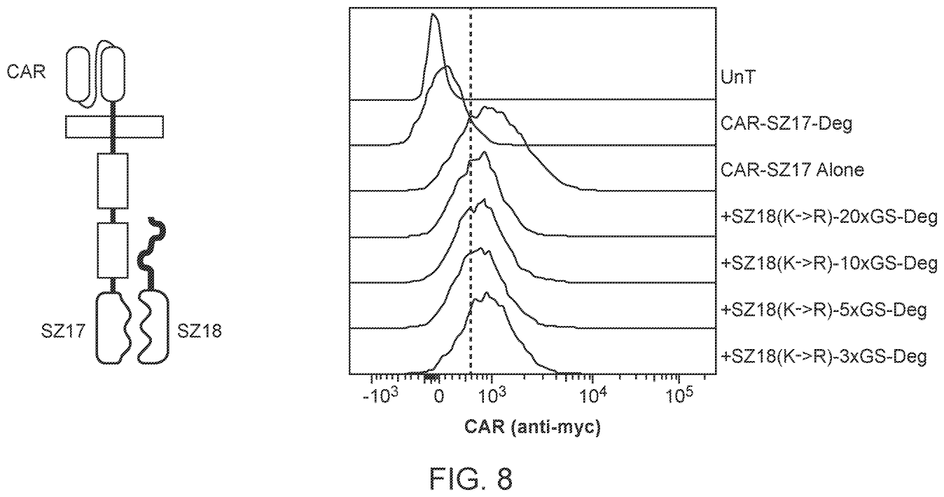

Next, the soluble STUD was used to target a membrane protein for degradation. The ability of the STUD to degrade a CAR in Jurkat cells was tested by generating a CAR construct with SynZIP17 fused to its C-terminus. However, while these STUDs worked to some extent, none of them were able to completely knockdown CAR expression (see FIG. 8). It was found that the Bonger degron, when directly fused to the CAR, was able to reduce CAR expression by over 90%. This result suggested that the soluble STUD was not working due to insufficient interaction with the CAR, rather than a defect with the ability of the degron to target membrane proteins for degradation.

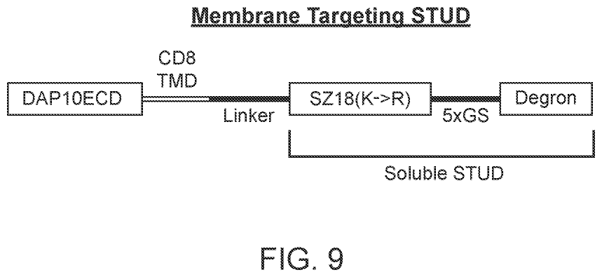

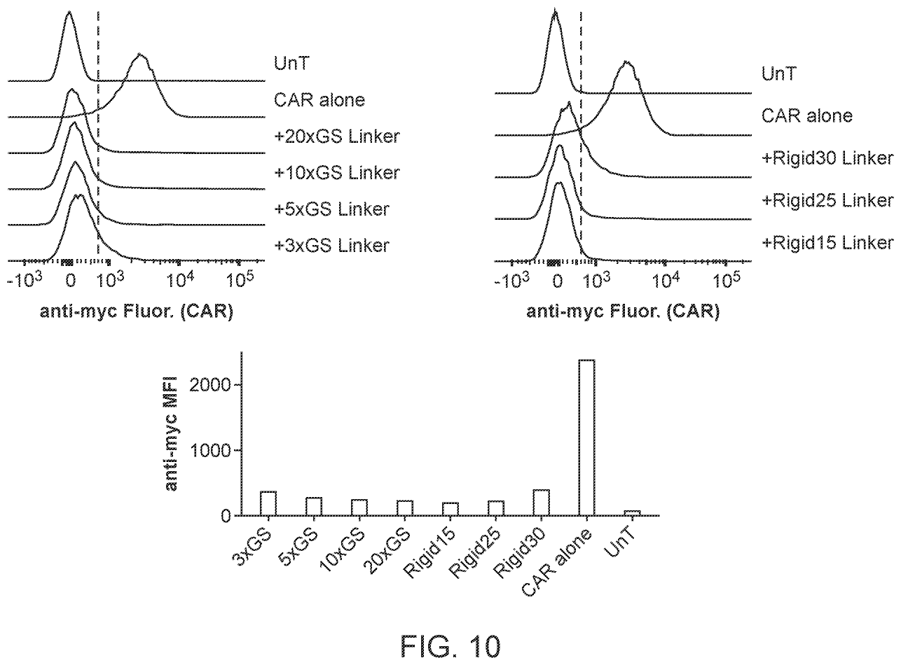

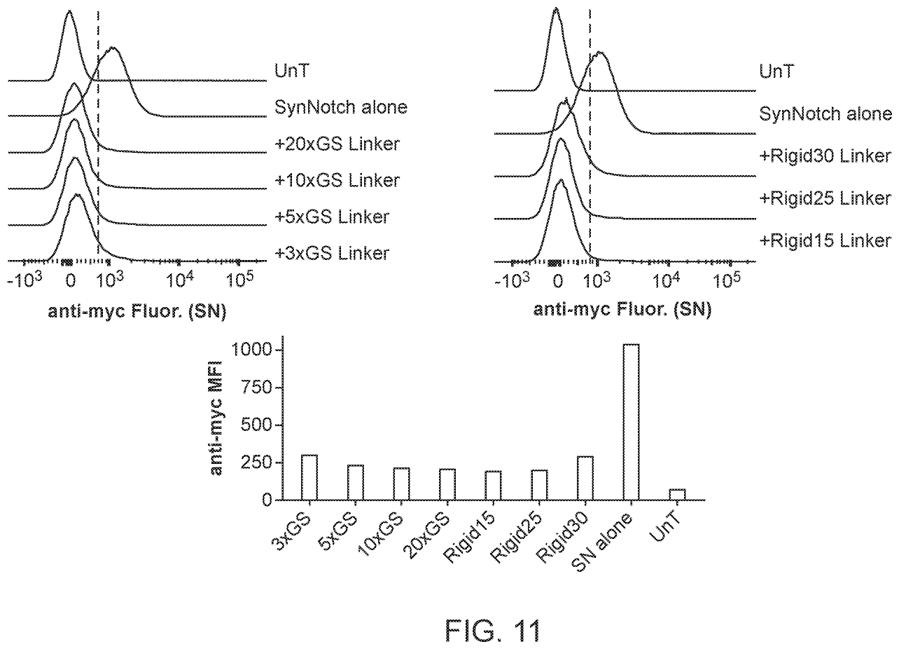

To increase the likelihood of interaction between the STUD and the CAR, a new STUD construct that was itself localized to the membrane using the DAP10 signal sequence was generated (FIG. 9). A library of linkers between the CD8 transmembrane domain and the soluble STUD was also tested. The ability of these new membrane targeting STUDs were tested for their ability to degrade both a CAR and a SynNotch in primary human CD4+ T cells. It was found that the best results were provided using a rigid linker between the CD8 TMD and STUD. All linkers were effective, but use of the Rigid15 linker resulted in over 95% knock-down of CAR expression as measured by surface staining for CAR expression (FIG. 10). This result was replicated for a membrane targeting STUD targeting a SynNotch for degradation (FIG. 11).

### Example 6

### NotchSTUD Receptor Design and Use

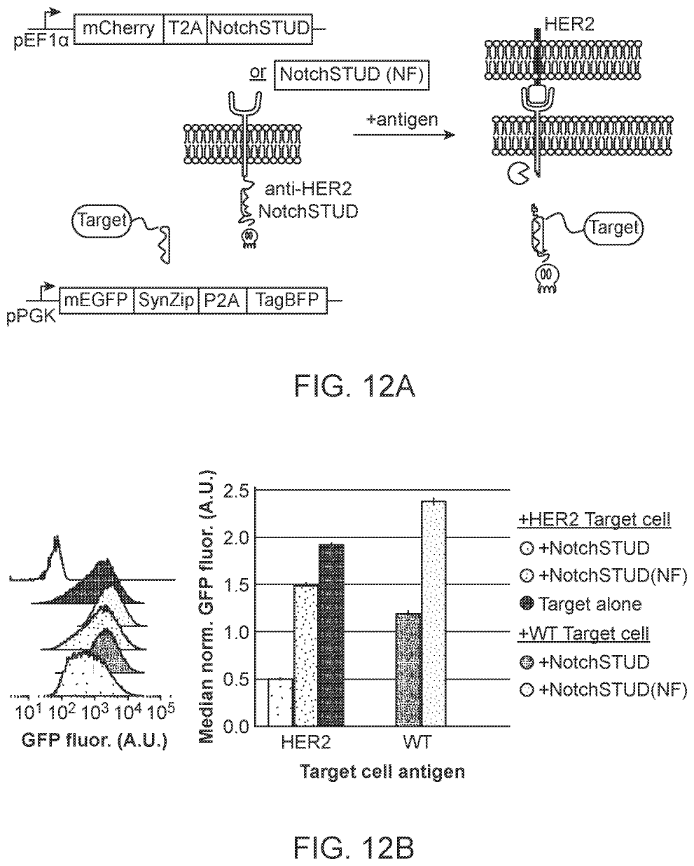

The system illustrated in FIG. 12(A) was designed an expressed in cells. This system contains a NotchSTUD synthetic receptor, which couples antigen binding to target degradation. Results from these experiments were shown in FIG. 12(B). Left: Representative flow cytometry distribution of target GFP fluorescence of cells gated on mCherry and tagBFP co-transduction markers 72 hours after coculture. Right: Quantification of median GFP target fluorescence normalized by tagBFP co-transduction marker following 72 hours of coculture with HER2+K562 target cells or WT K562 target cells. Black bars on bar plot represent standard deviation of three technical triplicates. NF=Non-functional.

### Materials and Methods

Cytosolic STUD for targeting GFP: Cytosolic STUDs were introduced by lentiviral transduction of two plasmids. The first encodes a green fluorescent protein (GFP) which will be a target for degradation alongside a BFP as a co-transduction marker. The second encodes the STUD protein, or non-functional controls, alongside an mCherry fluorescent protein as a co-transduction marker. Cells were then analyzed by flow cytometry. Cells were gated on expression of co-transduction fluorescent proteins (BFP/mCherry) and STUD efficacy was measured by knockdown of GFP fluorescence.

Using proteasome inhibitor to explore cytosolic GFP mechanism: To ascertain the mechanism by which the STUD degrades cytosolic GFP, we incubated cells with 5 μM of the proteasome inhibitor MG132 for 1 and 3 hours. Cells were then washed with PBS and analyzed by flow cytometry. Using the same 2-plasmid system as described above, we measured changes in GFP fluorescence relative to controls.

Membrane targeting STUD: Membrane targeting cells were introduced by lentiviral transduction of two plasmids. The first encodes a chimeric antigen receptor (CAR) or synthetic Notch (SynNotch) protein which will be a target for degradation alongside a BFP as a co-transduction marker. The second encodes the membrane localized STUD protein, or non-functional controls, alongside an mCherry fluorescent protein as a co-transduction marker. Cells were then analyzed by flow cytometry. Cells were gated on expression of co-transduction fluorescent proteins (BFP/mCherry) and STUD efficacy was measured by knockdown of CAR/SynNotch. CAR and SynNotch expression was measured by antibody staining for a peptide tag fused to the extracellular domain of the CAR/SynNotch.

### REFERENCES

1. Bonger, K. M., Chen, L.-C., Liu, C. W. & Wandless, T. J. Small-molecule displacement of a cryptic degron causes conditional protein degradation. Nat. Chem. Biol. 7, 531-537 (2011).
2. Daniel, K. et al. Conditional control of fluorescent protein degradation by an auxin-dependent nanobody. Nat. Commun. 9, 3297 (2018).
3. Arai, R., Ueda, H., Kitayama, A., Kamiya, N. & Nagamune, T. Design of the linkers which effectively separate domains of a bifunctional fusion protein. Protein Eng. 14, 529-532 (2001).
4. Wu, C. Y., Roybal, K. T., Puchner, E. M. & Onuffer, J. Remote control of therapeutic T cells through a small molecule-gated chimeric receptor. Science (2015).

1. A cleavable fusion protein comprising:
(a) an extracellular binding domain comprising a first protein binding domain, wherein the first protein binding domain specifically binds to a cell surface protein;
(b) a force sensing region;
(c) a transmembrane domain;
(d) one or more force-dependent cleavage sites that are cleaved when the force sensing region is activated; and
(e) an intracellular domain comprising:
i. a second protein binding domain, wherein the second protein binding domain specifically binds to a target protein; and
ii. a degradation domain, wherein the degradation domain is a degron or E3 ligase-recruiting domain, and
when the fusion protein is expressed in a mammalian cell, binding of the first binding domain to the cell surface protein induces proteolytic cleavage of the one or more force-dependent cleavage site to release the intracellular domain.

2. The fusion protein of claim 1, wherein the extracellular binding domain comprises a scFv, a nanobody, a cell-surface receptor or a ligand for a cell-surface receptor.

3. The fusion protein of claim 1, wherein the force sensing region and/or the one or more force-dependent cleavage sites are from a Delta/Serrate/Lag2 (DSL) superfamily protein.

4. The fusion protein of claim 1, wherein the second protein binding domain is a scFv, a nanobody, or a non-antibody binding domain.

5. The fusion protein of claim 1, wherein the degradation domain is a degron.

6. (canceled)

7. A cell comprising the fusion protein of claim 1, or a nucleic acid encoding the same.

8. The cell of claim 7, wherein the cell further comprises the target protein.

9. The cell of claim 8, wherein the target protein is localized to the plasma membrane.

10. The cell of claim 8, wherein the target protein is a transmembrane protein.

11. The cell of claim 8, wherein the target protein is associated with a transmembrane protein.

12. (canceled)

13. The cell of claim 8, wherein the target protein is an immune receptor.

14. The cell of claim 8, wherein the target protein is not localized to the plasma membrane.

15. (canceled)

16. The cell of claim 8 any of claims 8, 14 and 15, wherein the target protein is a transcription factor.

17. (canceled)

18. A protein circuit comprising
i. a fusion protein of claim 1; and
ii. the target protein;
wherein binding of the first binding moiety of the fusion protein to the cell surface protein on another cell releases the intracellular domain and alters degradation of the target protein.

19. The protein circuit of claim 18, wherein the target protein is localized to the plasma membrane and wherein binding of the first binding moiety of the fusion protein to the cell surface protein on another cell decreases degradation of the target protein.

20. The protein circuit of claim 18, wherein the target protein is not localized to the plasma membrane and wherein binding of the first binding moiety of the fusion protein to the cell surface protein on another cell increases degradation of the target protein.

21. A method for modulating degradation of a target protein, comprising:
introducing a first cell to a second cell, wherein:
(a) the first cell comprises:
i. the fusion protein of claim 1; and
ii. the target protein; and
(b) the second cell comprises the cell surface protein on its surface,
wherein binding of the first cell to the second cell via the fusion protein releases the intracellular domain, thereby altering degradation of the target protein.

22. (canceled)

23. The method of claim 21, wherein the target protein is localized to the plasma membrane and the introducing step results in a decrease in degradation of the target protein.

24. The method of claim 21, wherein the target protein is a chimeric antigen receptor.

25. The method of claim 21, wherein the target protein is not localized to the plasma membrane and the introducing step results in an increase in degradation of the target protein.


SEQUENCE LISTING (52 sequences)

SEQ ID NO: 1 - protein, 23 aa, Artificial Sequence
  synthetic sequence
HLMYVAAAAFVLLFFVGCGVLLS

SEQ ID NO: 2 - protein, 23 aa, Artificial Sequence
  synthetic sequence
RGKRGGHATNYRIVAPRSRDERG

SEQ ID NO: 3 - protein, 23 aa, Artificial Sequence
  synthetic sequence
ESLSAIEAELEKVAHQLQALRRG

SEQ ID NO: 4 - protein, 23 aa, Artificial Sequence
  synthetic sequence
KISGGPHISYPPLHEWVLREGEE

SEQ ID NO: 5 - protein, 23 aa, Artificial Sequence
  synthetic sequence
KISGGPHISYPPLHEWVLREGAA

SEQ ID NO: 6 - protein, 24 aa, Artificial Sequence
  synthetic sequence
LRELDLAQEKTGWFALGSRSHGTL

SEQ ID NO: 7 - protein, 24 aa, Artificial Sequence
  synthetic sequence
LRELDLAQEKTGWFALGSRHCGRT

SEQ ID NO: 8 - protein, 23 aa, Artificial Sequence
  synthetic sequence
YKKAGSGIPLRMNSLFRKRNKGK

SEQ ID NO: 9 - protein, 23 aa, Artificial Sequence
  synthetic sequence
VFSDLKNESGQEDKKRLLLGLDR

SEQ ID NO: 10 - protein, 22 aa, Artificial Sequence
  synthetic sequence
VFSDLKNESGQEDKKRLLLGLD

SEQ ID NO: 11 - protein, 23 aa, Artificial Sequence
  synthetic sequence
DGEDEGYFQELLGSVNSLLKELR

SEQ ID NO: 12 - protein, 22 aa, Artificial Sequence
  synthetic sequence
DGEDEGYFQELLGSVNSLLKEL

SEQ ID NO: 13 - protein, 23 aa, Artificial Sequence
  synthetic sequence
LEKEKLQARQQQLLKKIEHARTC

SEQ ID NO: 14 - protein, 20 aa, Artificial Sequence
  synthetic sequence
LEKEKLQARQQQLLKKIEHA

SEQ ID NO: 15 - protein, 23 aa, Artificial Sequence
  synthetic sequence
GPGPNGQRFCINSAALKFKPRKH

SEQ ID NO: 16 - protein, 23 aa, Artificial Sequence
  synthetic sequence
LRNAQMKNAIRKLCSRKAISSVK

SEQ ID NO: 17 - protein, 23 aa, Artificial Sequence
  synthetic sequence
LRNAQMKNAIRKLCSRKAISSDK

SEQ ID NO: 18 - protein, 23 aa, Artificial Sequence
  synthetic sequence
RRSCNDCQQMIVKLGGGTAIAAS

SEQ ID NO: 19 - protein, 23 aa, Artificial Sequence
  synthetic sequence
RRSCNDCQQMIVKLGGGTAIADS

SEQ ID NO: 20 - protein, 23 aa, Artificial Sequence
  synthetic sequence
QKSRKVDSKSLFHYRQYSAGKAA

SEQ ID NO: 21 - protein, 23 aa, Artificial Sequence
  synthetic sequence
QKSRKVDSKSLFHYRQYSAGKDD

SEQ ID NO: 22 - protein, 28 aa, Artificial Sequence
  synthetic sequence
QKSRKVDSKSLFHYRQYSAGKAAKASTN

SEQ ID NO: 23 - protein, 23 aa, Artificial Sequence
  synthetic sequence
REIQGIFFKEDSHKESNDCSCGG

SEQ ID NO: 24 - protein, 23 aa, Artificial Sequence
  synthetic sequence
SLTDVALEHHEECDCVCRGSTGG

SEQ ID NO: 25 - protein, 23 aa, Artificial Sequence
  synthetic sequence
RRLDGKEEDEKMSRASDRFRGLR

SEQ ID NO: 26 - protein, 19 aa, Artificial Sequence
  synthetic sequence
TRGVEEVAEGVVLLRRRGN

SEQ ID NO: 27 - protein, 5 aa, Artificial Sequence
  synthetic sequence
NLGIR

SEQ ID NO: 28 - protein, 10 aa, Artificial Sequence
  synthetic sequence
QRKLQRTSRG

SEQ ID NO: 29 - protein, 10 aa, Artificial Sequence
  synthetic sequence
QRKLQRTSRA

SEQ ID NO: 30 - protein, 12 aa, Artificial Sequence
  synthetic sequence
PHKRLLKGSQYG

SEQ ID NO: 31 - protein, 18 aa, Artificial Sequence
  synthetic sequence
TRGVEEVAEGVVLLRRRG

SEQ ID NO: 32 - protein, 6 aa, Artificial Sequence
  synthetic sequence
TRRRGN

SEQ ID NO: 33 - protein, 4 aa, Artificial Sequence
  synthetic sequence
RRRG

SEQ ID NO: 34 - protein, 115 aa, Artificial Sequence
  synthetic sequence
QIFVKTLTGKTITLEVESSDTIDNVKSKIQDKEGIPPDQQRLIFAGKQLEDGRTLSDYNIQKESTLHLVLRLRGGRHGSGAWLLPVSLVRRRTTLAPNTQTASPRALADSLMQRS

SEQ ID NO: 35 - protein, 21 aa, Artificial Sequence
  synthetic sequence
DDLLLPQDVEEFFEGPSEALR

SEQ ID NO: 36 - protein, 7 aa, Artificial Sequence
  synthetic sequence
ALAPYIP

SEQ ID NO: 37 - protein, 14 aa, Artificial Sequence
  synthetic sequence
KFERQKILDQRFFE

SEQ ID NO: 38 - protein, 21 aa, Artificial Sequence
  synthetic sequence
PLSSSVPSQKTYQGSYGFRLG

SEQ ID NO: 39 - protein, 4 aa, Artificial Sequence
  synthetic sequence
GSYG

SEQ ID NO: 40 - protein, 10 aa, Artificial Sequence
  synthetic sequence
DVQKADVSST

SEQ ID NO: 41 - protein, 8 aa, Artificial Sequence
  synthetic sequence
SPDSSTSP

SEQ ID NO: 42 - protein, 4 aa, Artificial Sequence
  synthetic sequence
RRRG

SEQ ID NO: 43 - protein, 5 aa, Artificial Sequence
  synthetic sequence
DINNN

SEQ ID NO: 44 - protein, 10 aa, Artificial Sequence
  synthetic sequence
GRRRRELDPM

SEQ ID NO: 45 - protein, 9 aa, Artificial Sequence
  synthetic sequence
RQRRELDPM

SEQ ID NO: 46 - protein, 8 aa, Artificial Sequence
  synthetic sequence
KIEAVKSE

SEQ ID NO: 47 - protein, 8 aa, Artificial Sequence
  synthetic sequence
KIEAVQSE

SEQ ID NO: 48 - protein, 8 aa, Artificial Sequence
  synthetic sequence
VGCGVLLS

SEQ ID NO: 49 - protein, 7 aa, Artificial Sequence
  synthetic sequence
GCGVLLS

SEQ ID NO: 50 - protein, 12 aa, Artificial Sequence
  synthetic sequence
  X (3)..(4): X can be any amino acid
  X (6)..(7): X can be any amino acid
  X (9)..(10): X can be any amino acid
HEXXHXXGXXHD

SEQ ID NO: 51 - protein, 4 aa, Artificial Sequence
  synthetic sequence
RRRG

SEQ ID NO: 52 - protein, 14 aa, Artificial Sequence
  synthetic sequence
GSGSGSGSGSRRRG